(19) United States
(12) Patent Application Publication
(10) Pub. No.: US 2013/0178862 A1
(43) Pub. Date: Jul. 11, 2013

(54) Intervertebral Implant Insertion Device and Method

(71) Applicant: Javier Garcia-Bengochea, Jacksonville, FL (US)

(72) Inventor: Javier Garcia-Bengochea, Jacksonville, FL (US)

(21) Appl. No.: 13/737,589

(22) Filed: Jan. 9, 2013

Related U.S. Application Data
(60) Provisional application No. 61/584,528, filed on Jan. 9, 2012.
(60) Provisional application No. 61/584,486, filed on Jan. 9, 2012.

(51) Int. Cl. A61F 2/46

(52) U.S. Cl. CPC A61F 2/4611
USPC 606/99

(57) ABSTRACT

An implant insertion device and its method of use, the device having a pair of opposing elongated blades retained by a housing. The distal ends of the blades are adapted to be inserted into the vertebral interspace. The housing comprises an internally threaded bore. An insertion rod having an elongated threaded rod passes through the housing and between the blades, whereby rotation of the insertion rod relative to the housing causes it to advance within the housing. An implant is temporarily mounted to the end of the insertion rod. As the insertion rod is advanced, the implant forces the distal ends of the blades apart such that proper placement of the implant in the intervertebral space is readily achieved. Once the implant is properly positioned free of the blades, the device is removed.

This application claims the benefit of U.S. Provisional Patent Application Ser. Nos. 61/584,528 and 61/584,486, both filed Jan. 09, 528, the disclosures of which are incorporated herein by reference.

### BACKGROUND OF THE INVENTION

This invention relates generally to the field of instruments and methods for the insertion of intervertebral implants, such implants being spacer members of sufficient strength to maintain separation between adjoining vertebrae during load, and typically being provided with cavities to receive bone graft material.

Over the past 15 years posterior approaches to anterior column reconstruction have become increasingly refined and popular. While anterior approaches have become more routine and lateral approaches have become excellent alternatives to anterior reconstruction of the spine, the posterior approach remains unparalleled in its versatility and comprehensive management of spinal pathology.

Heretofore the greatest challenges faced in posterior reconstruction of the spine have been the ability to place sufficiently large (i.e., wide) implants around the nerves and cauda equine as well as the ability to atraumatically distract the interspace with a solid implant impacted into the interspace between vertebrae. The shortcomings of the latter include damage to the vertebral endplates as well as loss of graft material from within the chambers of the implant.

Recently, traditional PLIF (posterior lumbar interbody fusion) and TLIF (transforaminal lumbar interbody fusion) implants have been supplanted by expandable cages that are placed into the interspace as collapsed entities and are opened or expanded once inside the disc space. The shortcomings of such devices include the inability to fully expand the device once in the disc space as well as the “gaps” created between the host bone (the vertebral endplate) and the graft material contained within the implant once the implant is expanded.

In conventional PLIF or TLIF surgery, the implant is packed with graft material and then impacted into the interspace with a mallet. In most instances the graft that was packed into the implant is dislodged from within the graft chamber of the implant by the impaction forces. This leads to “gaps” and may be a source of pseudoarthrosis or failure to achieve fusion.

In an effort to mitigate such effects, it is an object of this invention to provide an implant insertion device and method wherein the implant is gradually advanced into the vertebral interspace.

### SUMMARY OF THE INVENTION

In general, the invention is an implant insertion device and its method of use comprising a pair of opposing elongated blades retained by a housing. The distal ends of the blades are adapted to be inserted into the vertebral interspace. The housing comprises an internally threaded bore. An insertion rod comprising an elongated threaded rod passes through the housing and between the blades, whereby rotation of the insertion rod relative to the housing causes it to advance within the housing. An implant is mounted to the end of the insertion rod. As the insertion rod is advanced, the implant forces the blades apart such that proper placement of the implant in the intervertebral space is readily achieved. Once the implant is properly positioned free of the blades, the device is removed.

### BRIEF DESCRIPTION OF THE DRAWINGS

FIG. 1 is a view, partially in cross-section, of an embodiment of the invention.

FIG. 2 is a partial view of the embodiment of FIG. 1, wherein the implant has been advanced sufficient to spread the distal ends of the blades.

FIG. 3 is a partial view of one of the blades.

FIG. 4 is an end view of one of the blades.

### DETAILED DESCRIPTION OF THE INVENTION

With reference now to the drawings, several embodiments of the invention will be described. In general, the invention is an implant insertion device and its method of use comprising a pair opposing elongated blades 11 retained by a housing 21. The distal ends 12 of the blades 11 are adapted to be inserted into the vertebral interspace. The housing 21 comprises an internally threaded bore 24. An insertion rod 31 comprising an elongated threaded portion 32 passes through the housing 21 and between the blades 11, whereby rotation of the insertion rod 33 relative to the housing 21 causes it to advance within the housing 21. An implant 99 is temporarily mounted to the end of the insertion rod 31. As the insertion rod 31 is advanced, the implant 99 forces the blades 11 apart such that proper placement of the implant 99 in the intervertebral space is readily achieved. Once the implant 99 is properly positioned free of the blades 11, the device is removed.

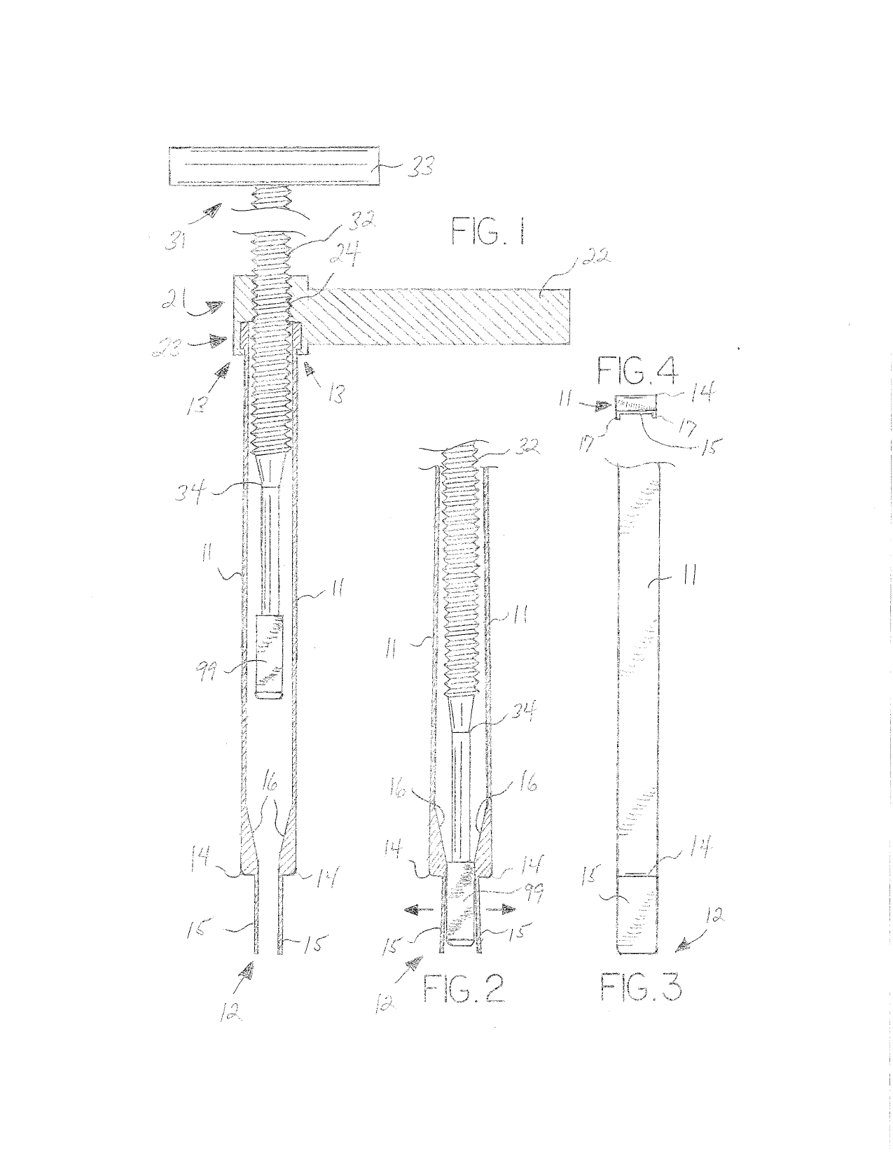

As shown in FIG. 1, one embodiment of the device comprises a pair of opposing elongated blades 11 each having a distal end 12 and a proximal end 13. The blades 11 are generally flat or slightly curved, and may be provided with linear rails 17 to retain and guide the implant 99 as it is advanced. The proximal ends 13 of the blades 11 are mounted within a housing 21, either fixedly or removably using connecting mechanisms 23 of known type. The blades 11 are formed of a material and shaped such that each is sufficiently rigid to allow for insertion of the distal end 12 into the intervertebral space yet is also sufficiently flexible so as to bend outwardly as the implant 99 is advanced, as shown in FIG. 2. Alternatively, the blades 11 may be mounted within housing 21 in a pivoting manner. Housing 21 is provided with a grip or handle 22. Housing 21 further comprises an internally threaded bore 24, and said blades 11 are positioned on opposite sides of said internally threaded bore 24.

In a preferred embodiment, the distal ends of each blade 11 comprises a thin insertion tip 15 and an external shoulder 14 that precludes insertion of the tips 15 into the intervertebral space beyond that which is needed or desired. The blades 11 may be inclined inwardly in the distal direction relative to each other or ramping surfaces 16 may be provided on one or both of the blades 11. The blades 11 may also be curved, bent or angled such that they are nearer to each other at the distal end 12.

The insertion rod 31 comprises a handle 33 mounted onto an elongated threaded portion 32 properly sized and adapted to be received within and cooperate with the internally threaded bore 24 of the housing 21. Such that rotation of the insertion rod 31 within the housing 21 results in movement of the insertion rod 31 in the axial direction relative to the housing 21. The implant 99 is temporarily mounted to the distal end of the insertion rod 31 such that it will be advanced between the blades 11 in the distal direction. The implant 99 may be structured with a cylindrical socket to receive a cylindrical post on the distal end of the insertion rod 31 such that the implant 99 does not rotate when the insertion rod 31 is rotated. Alternatively, free-rotational or swivel mechanism 34 may be provided in the insertion rod 31 such that the implant 99 does not rotate when the insertion rod 31 is rotated.

As seen best in FIG. 2, advancement of the implant 99 spreads the distal ends 12 of the blades 11, such that less resistance is encountered as the implant 99 is advanced beyond the distal ends 12 of the blades 11. Once the implant 99 is advanced free of the blades 11 into its desired position, the device is withdrawn from the intervertebral space.

It is understood that equivalents and substitutions for certain elements and structures set forth above may be obvious to those of skill in the art, and therefore the true scope and definition of the invention is to be as set forth in the following claims.

I claim:

1. An intervertebral implant insertion device comprising:
a housing comprising an internally threaded bore;
a pair of opposing elongated blades, each of said blades having a distal end and a proximal end, wherein said proximal ends of said blades are mounted to said housing on opposite sides of said internally threaded bore;
an insertion rod comprising a handle and an externally threaded portion, said insertion rod being disposed within said housing;
whereby rotation of said insertion rod relative to said housing advances an implant mounted onto said insertion rod toward and past said distal ends of said blades, and whereby said distal ends of said blades are spread apart upon advancement of said implant.

2. The device of claim 1, wherein said distal ends of said blades each comprises a insertion tip and an external shoulder.

3. The device of claim 1, wherein each of said blades comprises a ramping surface.

4. The device of claim 2, wherein each of said blades comprises a ramping surface.

5. The device of claim 2, wherein each of said blades comprises guide rails.

6. The device of claim 3, wherein each of said blades comprises guide rails.

7. The device of claim 4, wherein each of said blades comprises guide rails.

8. The device of claim 1, wherein said blades are fixedly mounted to said housing.

9. The device of claim 1, wherein said blades are removably mounted to said housing.

10. The device of claim 1, wherein said insertion rod further comprises a swivel mechanism such that said implant does not rotate when said insertion rod is rotated.

11. A method of positioning an intervertebral implant comprising the steps of:
providing an intervertebral implant insertion device comprising:
a housing comprising an internally threaded bore;
a pair of opposing elongated blades, each of said blades having a distal end and a proximal end, wherein said proximal ends of said blades are mounted to said housing on opposite sides of said internally threaded bore;
an insertion rod comprising a handle and an externally threaded portion, said insertion rod being disposed within said housing;
whereby rotation of said insertion rod relative to said housing advances an implant mounted onto said insertion rod toward and past said distal ends of said blades, and whereby said distal ends of said blades are spread apart upon advancement of said implant;
mounting an intervertebral implant to said insertion rod between said blades;
inserting said distal ends of said blades into an intervertebral space;
rotating said insertion rod relative to said housing such that said implant advances between said blades and spreads said distal ends of said blades apart;
continuing to advance said implant beyond said distal ends of said blades;
and removing said intervertebral implant insertion device from said intervertebral space.
